(19) United States
(12) Patent Application Publication
(10) Pub. No.: US 2023/0220029 A1
(43) Pub. Date: Jul. 13, 2023

(54) ANTIVIRAL BIOMIMETIC PEPTIDES AND USES THEREOF

(71) Applicant: VIRGINIA COMMONWEALTH UNIVERSITY, Richmond, VA (US)

(72) Inventor: Michael H. PETERS, Richmond, VA (US)

(21) Appl. No.: 17/997,038

(22) PCT Filed: Mar. 26, 2021
(86) PCT No.: PCT/US21/24319

§ 371 Date: Oct. 25, 2022

Related U.S. Application Data
(60) Provisional application No. 63/006,234, filed on Apr. 7, 2020.

(51) Int. Cl. C07K 14/515; A61P 31/14

(52) U.S. Cl. CPC C07K 14/515; A61P 31/14

(57) ABSTRACT

Antiviral peptides having sequence identity to a portion of ACE2 are provided. The peptides are useful for inhibiting coronavirus particle attachment to cells and thus are used for the treatment of coronavirus infections.

### FIELD OF THE INVENTION

The invention is generally related to biomimetic peptides that bind coronavirus spike protein and inhibit attachment of the virus to cells.

### BACKGROUND OF THE INVENTION

Numerous studies have shown that entry of enveloped viruses into host cells requires membrane fusion between virus and host cell. For most animal viruses, this fusion function is mediated by a single envelope glycoprotein on virions. The S protein has been shown to be the fusion protein that mediates cellular entry for coronavirus. See Spaan et al., J. Gen. Virol. 69:2939 (1988).

Like its predecessor coronaviruses, SARS-CoV-2 has a characteristic Spike protein (S) on its surface that embellishes both a prefusion state and fusion state. Each SARS-CoV-2 virion carries approximately 100 spike proteins per virion (Bar-On et al, 2020, eLife). The prefusion Spike protein (S) is a large trimeric protein where each protomer may be in a so-called “Up-state” or “Down-state”, depending on the configuration of its receptor binding domain (RBD). In the so-called “Up-state” of the RBD, the (prefusion) protein is able to bind to human epithelial cells expressing ACE2 (Angiotensin Converting Enzyme 2) on their surface and infect via a transformation to its fusion state. These cells include Type I and II pneumocytes; alveolar macrophage; nasal mucosal cells; and a large range of vascular epithelial cells throughout the body (kidney, heart, etc.). In the “Down-state” of the RBD the Spike protein is believed to be inactive or reduced in level to ACE2 binding and to cellular infection. Each Spike protein is believed to require at least one RBD in the so-called “Up-state” (Wrapp et al., Science, 367, 2020).

Active agents that can prevent viral attachment to cells and thus prevent or treat coronavirus infection are needed.

### SUMMARY

Described herein are helical peptide biomimetics from the Spike protein binding partner hACE2 that demonstrate strong and stable binding to the RBD thus reducing viral cell entry and systemic infection.

An aspect of the disclosure provides an antiviral peptide comprising an amino acid sequence at least 80% identical to SEQ ID NO: 4, wherein the peptide has at least one mutation relative to SEQ ID NO: 3, the naturally occurring sequence. In some embodiments, the amino acid sequence is selected from the group consisting of SEQ ID NO: 4, SEQ ID NO: 5, SEQ ID NO: 6, and SEQ ID NO: 7. In some embodiments, residue 20 is tyrosine. In some embodiments, the peptide is less than 30 amino acids in length. In some embodiments, the peptide is in a helical conformation. In some embodiments, the peptide is conjugated to a fatty acid.

Another aspect of the disclosure provide a miniprotein comprising an antiviral peptide as described herein. In some embodiments, the miniprotein is less than 100 amino acids in length.

Another aspect of the disclosure provides a pharmaceutical composition comprising an antiviral peptide as described herein and a pharmaceutically acceptable carrier.

Another aspect of the disclosure provides a method of treating a coronavirus infection in a subject in need thereof, comprising administering to the subject a therapeutically effective amount of an antiviral peptide as described herein. In some embodiments, the coronavirus infection is caused by SARS-Cov-2. In some embodiments, the peptide is administered subcutaneously or intranasally.

Another aspect of the disclosure provides a method of inhibiting coronavirus particle attachment to cells, comprising contacting the coronavirus particles with an effective amount of an antiviral peptide as described herein. In some embodiments, the coronavirus particles are SARS-Cov-2 particles.

### BRIEF DESCRIPTION OF THE DRAWINGS

FIGS. 1A-B. (A) a 25 residue helix abstracted from ACE2 in a pocket of the RBD. (B) Dominant Energetic Mappings or “Glue Points” of the Interaction of the RBD of SARS-CoV-2 with ACE2.

FIGS. 2A-B. (A) Partial charge interactions of the un-optimized 25 residue helix with the RBD of SARS-CoV-2. (B) Example of significantly improved interactions after optimization. Some mutated residues are associated with improved helical stability not shown. In addition, mutated residues must not decrease solubility.

FIGS. 3A-B. (A) Graphical wheel representation of the 23-residue optimized peptides (solubility/stability/helicity/binding). (B) The plot shows specific energetic contact residues, which are spaced 3 to 4 residues apart consistent with the helical conformation (3.4 residues per turn).

FIGS. 4A-B. Surrogate virus test results for peptide 122023-C. FIG. 4A are results obtained in a first experiment and FIG. 4B are results obtained in a second experiment two months later using the same solubilized peptide. These studies demonstrate excellent stability of the 23 residue peptides (Table 1).

FIG. 5. In-vitro SARS-CoV-2 live viral test results for test compound A (122023-A Table 1).

### DETAILED DESCRIPTION

The disclosure provides compositions and methods that are useful for preventing and treating a coronavirus infection in a subject. More specifically, the invention provides peptides and conjugates and pharmaceutical compositions containing those peptides and conjugates that block fusion of a coronavirus, such as the SARS-CoV-2 virus, to a target cell. The peptides are ACE2 biomimetic/decoy peptides that bind strongly and stably to the SD1 segment of the coronavirus Spike protein and disrupt attachment/fusion to cells.

Embodiments of the disclosure provide an isolated antiviral peptide having between 7 and 50 amino acids, e.g. less than 30 amino acids, e.g. about 23 amino acids, where the peptide exhibits antiviral activity against a coronavirus, and where the peptide contains a sequence comprising at least 7, 10, 15, 20, 25, 30, 35, or 40 contiguous amino acids from angiotensin-converting enzyme 2 (ACE2) as set forth in SEQ ID NO: 1. In some embodiments, the peptide has a length of 15-35 amino acids, e.g. about 20-30 amino acids. In some embodiments, the peptide comprises a sequence as set forth in SEQ ID NO: 2 which corresponds to residues 21-45 of ACE2. In some embodiments, the peptide comprises a sequence as set forth in SEQ ID NO: 3 which corresponds to residues 22-44 of ACE2. In some embodiments, the peptide comprises a sequence as set forth in SEQ ID NO: 4, 5, 6, or 7 which corresponds to mutated derivatives of residues 22-44 of ACE2. In some embodiments, the peptide assumes a helical conformation.

By an “isolated peptide” is meant a presently disclosed peptide that has been separated from components that naturally accompany it. Typically, the peptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a presently disclosed peptide. An isolated peptide may be obtained, for example, by extraction from a natural source, by expression of a recombinant nucleic acid encoding such a peptide; or by chemically synthesizing the protein. Purity can be measured by any appropriate method, for example, column chromatography, polyacrylamide gel electrophoresis, or by HPLC analysis.

ACE2 is an enzyme attached to the cell membranes of cells located in the lungs, arteries, heart, kidney, and intestines. ACE2 lowers blood pressure by catalyzing the hydrolysis of angiotensin II (a vasoconstrictor peptide) into angiotensin (1-7) (a vasodilator). ACE2 counters the activity of the related angiotensin-converting enzyme (ACE) by reducing the amount of angiotensin-II and increasing Ang(1-7). ACE2 also serves as the cell surface receptor for coronaviruses via attachment of the prefusion spike protein. The peptides disclosed herein are biomimetics of ACE2 and thus bind coronavirus spike protein, preventing viral attachment and entry into cells.

In some embodiments, the peptide contains at least one amino acid substitution relative to the naturally occurring ACE2 sequence. The peptides of the disclosure also may comprise peptide sequences that exhibit 70% or more sequence identity, e.g. 70%, 75%, 80%, 85%, 90%, 91%, 92%, 93%, 94%, 95%, 96%, 97%, 98%, 99% or more sequence identity, e.g. as determined by BLAST or FASTA algorithms, with at least 7, 10, 15, 20, 25, 30, 35, or 40 contiguous amino acids from any of the sequences disclosed herein.

In some embodiments, the peptide contains a conserved amino acid substitution. Conserved amino acid substitutions involve replacing one or more amino acids in a peptide sequence with amino acids of similar charge, size, and/or hydrophobicity characteristics. Amino acids may be substituted with an amino acid within the same group according to the following categories: acid, basic, polar neutral, and non-polar neutral. For example a conservative mutation would include substitution of one small, neutral, non-polar amino acid such as alanine, glycine, isoleucine, leucine, proline, and valine for another. Similarly, mutation of a glutamic acid to an aspartic acid, or vice versa would also be considered a conservative mutation. Exchange of phenylalanine, tyrosine, and tryptophan for each other would be considered a conservative mutation. When only conserved substitutions are made, the resulting peptide retains the functionality of the unsubstituted peptide. In some embodiments, the peptide includes nonconserved substitutions, e.g. to enhance the efficacy of the peptide.

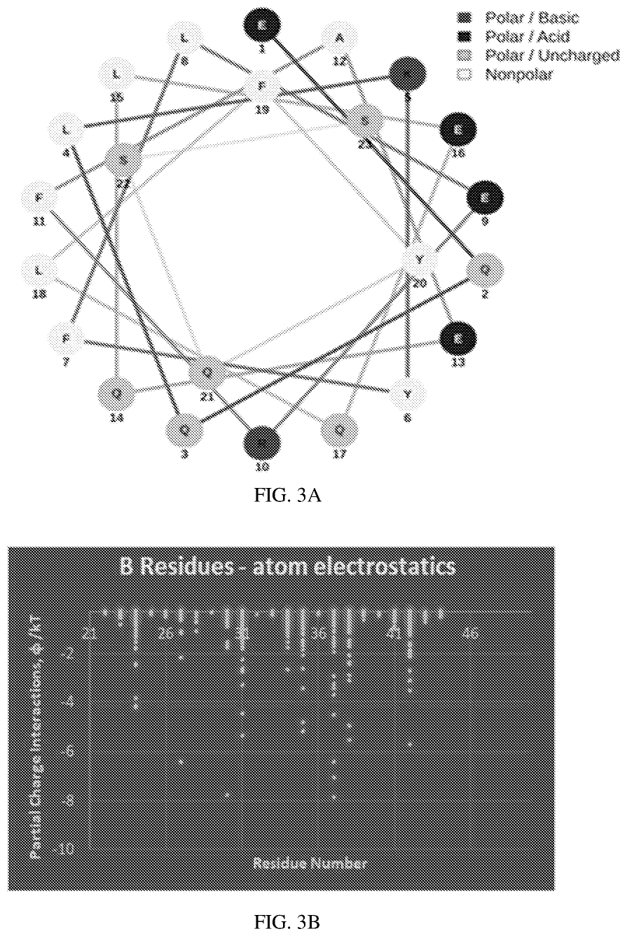

In some embodiments, in an exemplary 23 amino acid peptide, residues 1, 9, 13, and 16 are polar/acid residues; residues 2, 3, 14, 21, 21, and 23 are polar/uncharged residues; residues 4, 6, 7, 8, 11, 12, 15, 18, 19, and 20 are nonpolar residues; and residues 5 and 10 are polar/basic residues (see FIG. 3A). In some embodiments, residue 20 (corresponding to residue 41 of ACE2) is tyrosine. In addition, glutamic acid at position 1 and lysine at position 5 may form a “helical staple” and add stability to the helix. Thus, in some embodiments, these residues are conserved in the peptides disclosed herein. In some embodiments, the amino acid at position 2 is an amino acid other than glutamic acid which may break the staple and decrease solubility. In some embodiments, the amino acid at position 2 is glutamine which may recover the staple and improve solubility and binding. In some embodiments, the native pair phenyalanine at position 7 and phenyalanine at position 11 are conserved in order to enhance helicity.

In some embodiments, the peptide is conjugated or linked to a macromolecule or drug carrier, for example, lipid-fatty acid conjugates, polyethylene glycol, or carbohydrates. Fatty acid conjugation is obtained by fatty acid modification of the main chain structure or contralateral chain group of polypeptide drugs. Types of suitable fatty acids include, but are not limited to: Caprylic acid (C8), Capric acid (C10), Lauric acid (C12), Myristic acid (C14), Palmitic acid (C16), Stearic acid (C18) etc.

Ex vivo conjugation of the peptides to a macromolecule such as human serum albumin (HSA) produces a highly soluble conjugate that can be purified and administered in tightly controlled dosage. The cloaked conjugate is biologically active as the conjugate, i.e. it does not act as a prodrug that releases the peptide moiety from the conjugate and cleavage of the conjugate is not required for biological activity. In some embodiments, the peptide and the macromolecule are linked in an approximately 1:1 ratio, to avoid “haptenization” of the biologically active moiety and generation of an immune response to the conjugate. In some embodiments, more than one molecule of peptide is linked to the macromolecule, which may be achieved via a “multivalent” linker that is attached to a single point of the macromolecule. For example, a linker can be appended to C34 of HSA that permits attachment of a plurality of peptides to the linker. Multivalent linkers are known in the art and can contain, for example, a thiophilic group for reaction with C34 of HSA, and multiple nucleophilic (such as NH or OH) or electrophilic (such as activated ester) groups that permit attachment of a plurality of peptides to the linker.

Activated linkers that are particularly suited for linkage to thiols include unsaturated cyclic imides such as maleimides, α-halo esters, such as α-iodo- and α-bromo acetates, and vinyl pyridine derivative. Such linkers can be added to the peptides during synthesis and can be added at any point in the sequence although the N and/or C terminus advantageously is used. Suitable activated linkers are commercially available from, for example, Pierce Chemical (Rockford, Ill.). Methods for preparing suitable activated compounds for linking to HSA are known in art. See for example, U.S. Pat. No. 5,612,034, which is incorporated herein in its entirety.

Another blood component that is suitable for linkage to the anti-viral compounds is an immunoglobulin (“Ig”) molecule. An Ig refers to any suitable immunoglobulin or immunoglobulin derivative known in the art, and includes, for example, whole IgG, IgM, Fab fragments, F(ab′)2 fragments, and single chain Fv fragments.

Other blood components suitable for use in the present disclosure include transferrin, ferritin, steroid binding proteins, thyroxin binding protein, and α-2-macroglobulin.

In some embodiments, the peptides described herein are incorporated into miniproteins. Miniproteins are a group of protein scaffolds characterized by small (1-10 kDa) size, stability, and versatility in drug-like roles. Coming largely from native sources, they have been widely adopted into drug development pipelines. Various mini-proteins have been recently proposed as RBD binders to inhibit SARS-CoV-2 infections (Cao, L., et al, bioRxiv, doi.org/10.1101/2020.08.03.234914). Such miniproteins are less than about 100 residues in length, e.g. about 50-70 residues in length. In some embodiments, such miniproteins incorporate the mutated peptide sequences as described herein, thus yielding a nano- to pico-molar binder to SARS-CoV-2 spike protein.

In some embodiments, the peptides may be synthesized with additional chemical groups present at their amino and/or carboxy termini, such that, for example, the stability, bioavailability, and/or inhibitory activity of the peptides is enhanced. For example, hydrophobic groups such as carbobenzoxyl, dansyl, or t-butyloxycarbonyl groups, may be added to a peptide's amino terminus. Likewise, an acetyl group or a 9-fluorenylmethoxy-carbonyl group may be placed at a peptide's amino terminus. Additionally, a hydrophobic group, t-butyloxycarbonyl, or an amido group may be added to a peptide's carboxy terminus. Further, non-naturally occurring amino acids can be used to improve a peptide's stability, bioavailability, or binding/inhibitory characteristics. For example, methionine can be replaced with norleucine. Other non-naturally occurring amino acid residues are well known.

Further embodiments provide a nanoparticle or microparticle comprising peptide as described herein. The peptide may be loaded onto or into, or otherwise associated with the particle. In particular embodiments, the microparticle or nanoparticle comprises a biodegradable polymer or blends of polymers selected from the group consisting of poly(lactic-co-glycolic acid) (PLGA), poly(beta-amino ester) (PBAE), polycaprolactone (PCL), polyglycolic acid (PGA), polylactic acid (PLA), poly(acrylic acid) (PAA), poly-3-hydroxybutyrate (P3HB) and poly(hydroxybutyrate-co-hydroxyvalerate). In other embodiments, nondegradable polymers that are used in the art, such as polystyrene, are blended with a degradable polymer or polymers from above to create a copolymer system. Accordingly, in some embodiments, a nondegradable polymer is blended with the biodegradable polymer.

The peptides of the disclosure may be synthesized or prepared by techniques well known in the art. Peptide synthesizers are commercially available from, for example, Applied Biosystems or Milligen/Biosearch. See also, for example, Creighton, 1983, Proteins: Structures and Molecular Principles, W. H. Freeman and Co., N.Y. Short peptides, for example, can be synthesized on a solid support or in solution. Longer peptides, or fusions of longer peptides with carrier proteins such as human serum albumin, may be made using recombinant DNA techniques. Nucleotide sequences encoding the desired peptides or fusion proteins containing the peptides may be synthesized, and/or cloned, and expressed according to techniques well known to those of ordinary skill in the art. See, for example, Sambrook, et al., 1989, Molecular Cloning, A Laboratory Manual, Vols. 1-3, Cold Spring Harbor Press, N.Y.

The peptides also may be synthesized such that one or more of the bonds linking the amino acid residues of the peptides are non-peptide bonds. Alternative non-peptide bonds may be formed by reactions well known to those in the art, and may include, but are not limited to imino, ester, hydrazide, semicarbazide, and azo bonds.

Further embodiments provide a pharmaceutical composition comprising a peptide or composition as described herein and a pharmaceutically acceptable diluent, adjuvant and/or excipient.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Suitable pharmaceutically acceptable carriers include but are not limited to water, salt solutions, alcohols, gum arabic, vegetable oils, benzyl alcohols, polyethylene glycols, gelate, carbohydrates such as lactose, amylose or starch, magnesium stearate talc, silicic acid, viscous paraffin, perfume oil, fatty acid monoglycerides and diglycerides, pentaerythritol fatty acid esters, hydroxymethylcellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. Other suitable excipients include, for example an inert diluent such as lactose; granulating and disintegrating agents such as cornstarch; binding agents such as starch; and lubricating agents such as magnesium stearate.

The compositions may be provided in the form of tablets, coated tablets, dragees, hard or soft gelatin capsules, solutions, emulsions or suspensions.

The compositions of the present disclosure may also contain other components such as, but not limited to, antioxidants, additives, adjuvants, buffers, tonicity agents, bioadhesive polymers, and preservatives. It should be appreciated that the compositions of the present disclosure may be buffered by any common buffer system such as phosphate, borate, acetate, citrate, carbonate and borate-polyol complexes, with the pH and osmolality adjusted in accordance with well-known techniques to proper physiological values.

An additive such as a sugar, a glycerol, and other sugar alcohols, can be included in the compositions of the present disclosure. Pharmaceutical additives can be added to increase the efficacy or potency of other ingredients in the composition. For example, a pharmaceutical additive can be added to a composition of the present disclosure to improve the stability of the bioactive agent, to adjust the osmolality of the composition, to adjust the viscosity of the composition, or for another reason, such as effecting drug delivery. Non-limiting examples of pharmaceutical additives of the present disclosure include sugars, such as, trehalose, mannose, D-galactose, and lactose.

In an embodiment, if a preservative is desired, the compositions may optionally be preserved with any well-known system such as benzyl alcohol with/without EDTA, benzalkonium chloride, chlorhexidine, Cosmocil® CQ, or Dowicil 200.

Further embodiments provide a method of inhibiting coronavirus particle attachment to cells, e.g. epithelial cells, comprising contacting the coronavirus particles with an effective amount of an antiviral peptide or composition as described herein.

Further embodiments provide a method of treating or preventing a coronavirus infection in a subject, comprising administering to a patient suffering from (or suspected of suffering from) the infection a therapeutically effective amount of a peptide or composition as described herein.

By a “therapeutically effective amount” is meant a sufficient amount of active agent to treat the disease or disorder at a reasonable benefit/risk ratio applicable to any medical treatment. It will be understood that the total daily usage of the compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific active agent employed; and like factors well known in the medical arts. For example, it is well within the skill of the art to start doses of the compound at levels or frequencies lower than those required to achieve the desired therapeutic effect and to gradually increase the dosage or frequency until the desired effect is achieved. However, the daily dosage of the active agent may be varied over a wide range from 0.01 to 1,000 mg per adult per day. In particular, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, in particular from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day. In some embodiments, the composition is administered daily or 2, 3, 4 or more times weekly.

The peptides may be used as a prophylactic measure in previously uninfected individuals after acute exposure to a coronavirus. Examples of such prophylactic use of the peptides may include, but are not limited to, settings where the likelihood of viral transmission exists, such as, for example, in hospitals and transport termini such as airports and train stations. The peptides may thus serve the role of a prophylactic vaccine, wherein the host raises antibodies against the peptides of the invention, which then serve to neutralize viruses by, for example, inhibiting further infection. In some embodiments, antibodies raised against the peptides are administered as a treatment to a subject.

The peptides and compositions described herein may be used to treat or prevent an infection caused by any type of coronavirus, e.g. patients with a confirmed diagnosis of a coronavirus infection. Coronaviruses form the subfamily Orthocoronavirinae, which is one of two sub-families in the family Coronaviridae, order Nidovirales, and realm Riboviria. They are divided into the four genera: Alphacoronavirus, Betacoronavirus, Gammacoronavirus and Deltacoronavirus. Alphacoronaviruses and betacoronaviruses infect mammals, while gammacoronaviruses and deltacoronaviruses primarily infect birds. In some embodiments, the coronavirus is a Betacoronavirus selected from Betacoronavirus 1 (Bovine Coronavirus, Human coronavirus 0C43), Hedgehog coronavirus 1, Human coronavirus HKU1, Middle East respiratory syndrome-related coronavirus, Murine coronavirus, Pipistrellus bat coronavirus HKU5, Rousettus bat coronavirus HKU9, Severe acute respiratory syndrome-related coronavirus (SARS-CoV, SARS-CoV-2), and Tylonycteris bat coronavirus HKU4.

In some embodiments, the peptides have strong binding and inhibition for SARS-CoV-2 variants such as B.1.1.7 or B.1.351, or any variant carrying the N501Y mutation.

A patient or subject to be treated by any of the compositions or methods of the present disclosure can mean either a human or a non-human animal including, but not limited to dogs, horses, cats, rabbits, gerbils, hamsters, rodents, birds, bats, aquatic mammals, cattle, pigs, camelids, and other zoological animals. The subject may be an adult or pediatric patient.

Suitable routes of administration may include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections, etc.

In some embodiments, the compositions are delivered subcutaneously with the peptides being conjugated to fatty acids.

In some embodiments, the compositions are delivered as an inhalational drug product to the deep regions of the lung where the virion tends to maintain its highest concentration or viral load. A smaller particle size distribution (˜1-2 μm) required to reach the lower lung region also increases the likelihood of systemic absorption that could reduce multi-organ involvement that has been observed with SARS-CoV-2 infection, therefore reducing mortality associated with COVID-19 disease.

For injection, the peptides may be formulated in aqueous solutions, preferably in physiologically compatible buffers such as Hanks' solution, Ringer's solution, or physiological saline buffer. For such transmucosal administration, penetrants appropriate to the barrier to be permeated are used in the formulation. Such penetrants are generally known in the art. Other diluents, adjuvants, and excipients are known in the art.

The peptides may be formulated with a suitable adjuvant in order to enhance the immunological response. Such adjuvants may include, but are not limited to mineral gels such as aluminum hydroxide; surface active substances such as lysolecithin, pluronic polyols, polyanions; other peptides; oil emulsions; and potentially useful human adjuvants such as BCG and Corynebacterium parvum.

Further embodiments provide a kit containing some or all of the components, reagents, supplies, and the like to practice a method according to the presently disclosed subject matter. In one embodiment, a kit comprises at least one container (e.g. a vial, tube, or ampoule) comprising an isolated peptide.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### EXAMPLE

This study describes the development of novel peptide mimetics with improved stability and binding affinity for the ACE2-RBD interface to act as competitive inhibitors for the SARS-CoV-2 virions, decreasing viral cell entry and reducing replication.

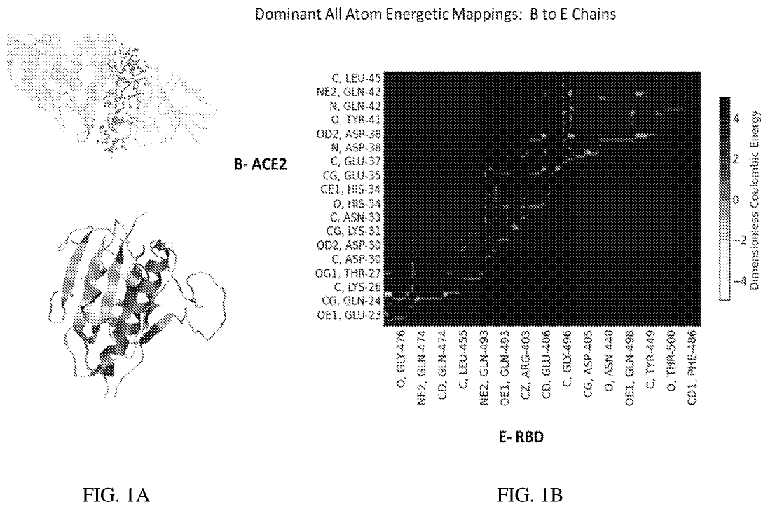

To initially generate optimized peptide mimetic candidates, our in-house energetic mapping algorithm (Krall et al, Proteins. Structure, Function and Bioinformatics, 82, 2253-2262, 2014) was used based on the published structure of the RBD of SARS-CoV-2 Spike protein (S) with hACE2 (PDB ID 6M17; Yan et al., Science, 2020). This mapping algorithm efficiently parses the strongest non-covalent atom-atom interactions and their inter-atomic distances from structure file data according to empirically established criteria based on the ab initio AMBER03 force field model to ensure that all dominant interactions are accounted. The native binding of hACE2 with SARS-CoV-2 RBD is shown in FIG. 1.

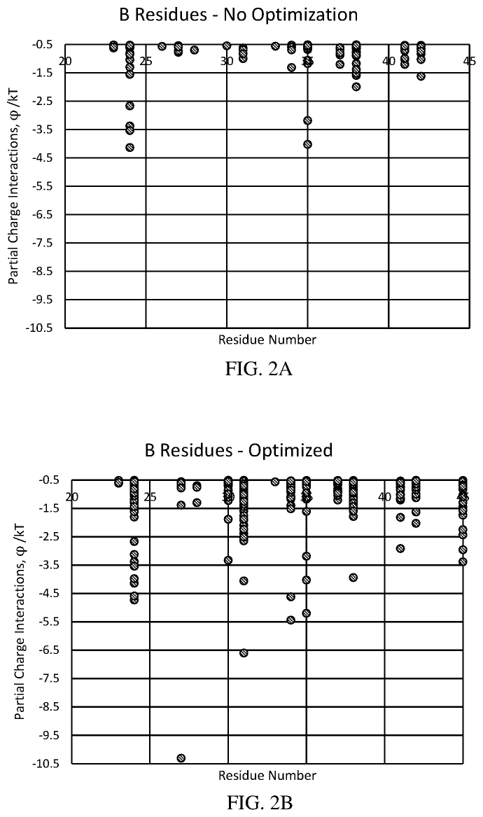

Specific energetic contact points (atom-atom interactions) along the binding interface for any given initial structure, or ensemble of structures from static structure files, and dynamic simulations were used to identify potential mutations for improved RBD-ACE2 binding (FIG. 2). Many of these energetic contact points are already in their lowest energetic state (including across ensembles) and mutations will not improve the local binding of those contacts. However, there are also clear, weaker energetic contact points across the binding interface, and these can be strategically mutated for improved binding (FIG. 3). The strongest energetic contact points are approximately 3 to 4 residues apart as consistent with the structure of alpha-helices which are characterized by approximately 3.4 residues per turn.

In general, optimization involves mutating specific residues to improve binding but including other critical, clinical translational aspects such as stability/helicity as well as solubility. In addition to the all-atom detailed energetic contact map, we studied the binding and stability of these purposefully mutated helical segments using computational dynamic simulations. Development of peptide biomimetics must consider the structural stability of parsed segments in addition to binding energies and entropies including, for example, acetylated N-terminal and amidated C-terminal capping.

In general, there are three primary constraints associated with optimization methods for the purposes of developing therapeutic peptides: (i) good water solubility, (ii) good binding, and (iii) good stability/helicity. Many times, these constraints are competing. For example, improved binding may result in poor water solubility and/or poor stability/helicity. So, optimizations methods are not straight forward.

Based on these constraints and our computational methods and analysis, we have developed hACE2 peptide mimetics with higher solubility, stability/helicity, and binding affinity for RBD of SARS-CoV-2 than the native ACE2 helical segment, and therefore are optimized inhibitors of viral infection (FIG. 3 and Table 1 below).

TABLE 1
Optimized 23 Residue Peptides

| | | SEQ |
|---|---|---|
| | | ID |
| Peptide | Sequence | NO. |
| Native | E E Q A K T F L D K F N H | 3 |
| | E A E D L F Y Q S S | |
| 122023-A | E Q Q L K Y F L E R F A E | 4 |
| | Q L E Q L F Y Q S S | |
| 122023-C | E Q Q L K Y F L E R F N E | 5 |
| | Q L E D L F Y Q S S | |
| 122023-D | E Q Q L K Y F L E R F X R | 6 |
| | Q L E Z L F Y Q S S | |
| 122023-E | E Q Q L K Y F L E R F X K | 7 |
| | Q L E Z L F Y Q S S | |

X is A, N, or L
Z is D or Q

Residue 41Y is necessary for strong binding to any SARS-CoV-2 variant carrying the N501Y mutation. Residue 41Y binds to N501 in the native state as shown in the energetic mappings. The N501Y mutation presents a potentially much stronger Tyrosine-Tyrosine interaction residue pair than Asparagine-Tyrosine (see, e.g., Miyazawa and Jernigan, J. Mol. Biology, 256, 1996).

### Results

Peptides are dissolved in PBS and stored with 10% glycerol in −20C. Optimized peptides showed excellent solubility in the temperature range of 20 to 30° C.

### SARS-CoV-2 Surrogate Virus ELISA

The Surrogate Virus Neutralization Test (sVNT) ELISA was developed to measure SARS-CoV-2 neutralizing antibodies from patient serum and can be readily adapted to measure IC50 values of designed peptides in a Biosafety Level 2 facility. This test has demonstrated >95% efficiency at detection of nAbs in COVID-19 patients (Tan et al., Nature Biotechnology, 38, 1073-1078, 2020). The two major components of the ELISA assay are (Horse Radish Peroxidase) HRP-conjugated-S-protein-RBD and hACE2 coated 96 well plates. In the absence of any nAbs or inhibitors, HRP-RBD binds to hACE2 and can be readily detected using absorbance at 450 nm. For positive control, we used a synthetic antibody, Fc-hACE2 which demonstrated an IC value of approximately 10 nanomolar and therefore is an excellent control for these ACE2 peptide mimetic studies. Negative control is no added inhibitor (solution buffer only) or nAbs human serum lacking antibodies to SARS-CoV-2. Percent inhibition is measured as the difference in mean OD values of patient serum or inhibitors to average negative control OD. Negative control in this case gives the highest absorbance signal in this assay:

% Inhibition=100[OD>_{Control}−<OD>_{Inhibitor}]/<OD>_{control}

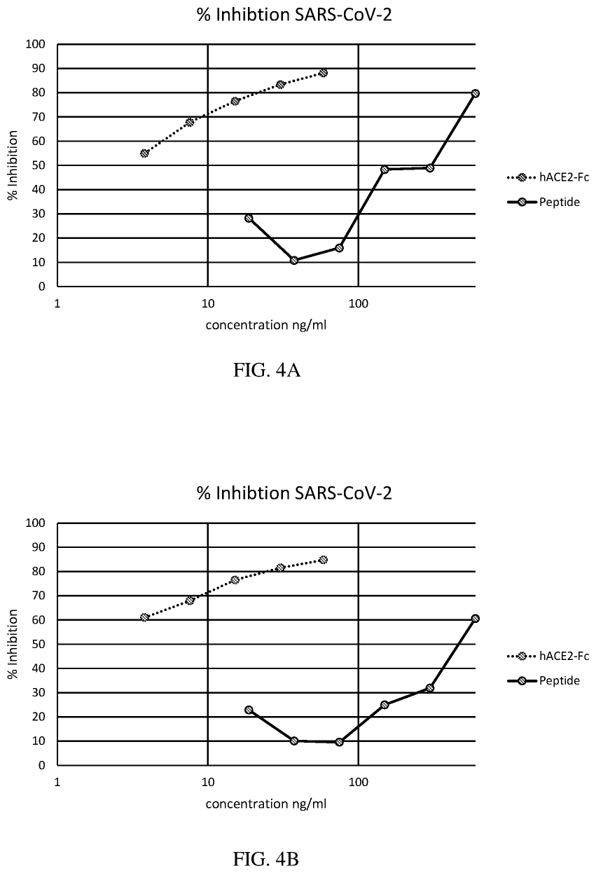

FIG. 4 shows results obtained for peptide 122023-C demonstrating an IC50 value of around 100 ng/ml in a dose-dependent fashion. Excellent peptide stability was demonstrated by conducting ELISA tests two months apart with the same inhibitor, as shown in FIG. 4A-B.

### SARS-CoV-2 Live Virus Testing

Test Article A was tested for antiviral activity against live SARS-CoV-2, including preexposure of virus to samples for 1 hour in serum-free media prior to adding to cell plates. SARS-CoV-2, USA-WA1/2020 (World Reference Center for Emerging Viruses and Arboviruses, WRCEVA), was tested in Vero E6 cells (ATCC) and test media was serum-free media of MEM (Cytiva) supplemented with 50 μg/mL gentamicin (Sigma). Cell culture media for plates containing cells was MEM with 2% FBS (Cytiva) and gentamicin. Virus was diluted in serum-free test media to achieve a MOI of 0.001 for antiviral tests.

Compounds were serially diluted using eight 2-fold dilutions in serum-free test media so that the highest test concentration on cell plates was 200 μM. Each dilution was added to 5 wells of a 96-well round bottom plate containing no cells. Three wells of each dilution were infected with virus, and two wells remained uninfected as toxicity controls. Six wells were infected and untreated as virus controls, and six wells were uninfected and untreated as cell controls. Sample dilutions and virus were incubated together for 1 hour at 37° C. prior to adding to cell plates. Remdesivir was tested in parallel as a positive control and treated in the same way as described here. Following the pre-exposure of sample and virus, plate contents (100 μL per well) were transferred to plates containing 80-100% confluent Vero E6 cells with 100 μL MEM with 4% FBS and 50 μg/mL gentamicin (final culture media was MEM with 2% FBS and gentamicin on plate). Plates were incubated at 37±2° C., 5% CO₂.

On day 4 post-infection, once untreated virus control wells reached maximum CPE, plates were stained with neutral red dye for approximately 2 hours (±15 minutes). Supernatant dye was removed and wells rinsed with PBS, and the incorporated dye was extracted in 50:50 Sorensen citrate buffer/ethanol for >30 minutes and the optical density was read on a spectrophotometer at 540 nm. Optical densities were converted to percent of cell controls and normalized to the virus control, then the concentration of test compound required to inhibit CPE by 50% (EC50) was calculated by regression analysis. The concentration of compound that would cause 50% cell death in the absence of virus was similarly calculated (CC50). The selective index (SI) is the CC50 divided by EC50.

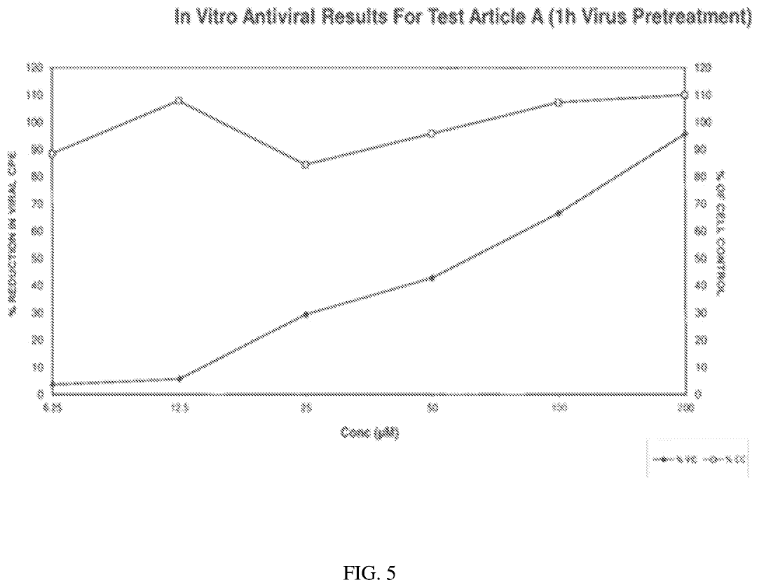

As shown in FIG. 5, Test compound A (122023-A Table 1) demonstrated good antiviral activity with an IC50 value in the low micromolar range (59 micromolar) and up to 95% inhibition at the highest concentration studied.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. An antiviral peptide comprising an amino acid sequence at least 80% identical to SEQ ID NO: 4, wherein the peptide has at least one mutation relative to SEQ ID NO: 3.

2. The antiviral peptide of claim 1, wherein the amino acid sequence is selected from the group consisting of SEQ ID NO: 4, SEQ ID NO: 5, SEQ ID NO: 6, and SEQ ID NO: 7.

3. The antiviral peptide of claim 1, wherein residue 20 is tyrosine.

4. The antiviral peptide of claim 1, wherein the peptide is less than 30 amino acids in length.

5. The antiviral peptide of claim 1, wherein the peptide is in a helical conformation.

6. The antiviral peptide of claim 1, wherein the peptide is conjugated to a fatty acid.

7. A miniprotein comprising the antiviral peptide of claim 1, wherein the miniprotein is less than 100 amino acids in length.

8. A pharmaceutical composition comprising the antiviral peptide of claim 1 and a pharmaceutically acceptable carrier.

9. A method of treating a coronavirus infection in a subject in need thereof, comprising administering to the subject a therapeutically effective amount of the antiviral peptide of claim 1.

10. The method of claim 9, wherein the coronavirus infection is caused by SARS-Cov-2.

11. The method of claim 9, wherein the amino acid sequence is selected from the group consisting of SEQ ID NO: 4, SEQ ID NO: 5, SEQ ID NO: 6, and SEQ ID NO: 7.

12. The method of claim 9, wherein residue 20 is tyrosine.

13. The method of claim 9, wherein the peptide is less than 30 amino acids in length.

14. The method of claim 9, wherein the peptide is in a helical conformation.

15. The method of claim 9, wherein the peptide is conjugated to a fatty acid.

16. The method of claim 9, wherein the peptide is incorporated into a miniprotein less than 100 amino acids in length.

17. The method of claim 9, wherein the peptide is administered subcutaneously or intranasally.

18. A method of inhibiting coronavirus particle attachment to cells, comprising contacting the coronavirus particles with an effective amount of the antiviral peptide of claim 1.

19. The method of claim 18, wherein the coronavirus particles are SARS-Cov-2 particles.

20. The method of claim 18, wherein the amino acid sequence is selected from the group consisting of SEQ ID NO: 4, SEQ ID NO: 5, SEQ ID NO: 6, and SEQ ID NO: 7.

21. The method of claim 18, wherein residue 20 is tyrosine.

22. The method of claim 18, wherein the peptide is less than 30 amino acids in length.

23. The method of claim 18, wherein the peptide is in a helical conformation.

24. The method of claim 18, wherein the peptide is conjugated to a fatty acid.

25. The method of claim 18, wherein the peptide is incorporated into a miniprotein less than 100 amino acids in length.


SEQUENCE LISTING (7 sequences)

SEQ ID NO: 1 - protein, 805 aa, Homo sapiens [sequence omitted]

SEQ ID NO: 2 - protein, 25 aa, Artificial Sequence
  Synthetic peptide
IEEQAKTFLDKFNHEAEDLFYQSSL

SEQ ID NO: 3 - protein, 23 aa, Artificial Sequence
  Synthetic peptide
EEQAKTFLDKFNHEAEDLFYQSS

SEQ ID NO: 4 - protein, 23 aa, Artificial Sequence
  Synthetic peptide
EQQLKYFLERFAEQLEQLFYQSS

SEQ ID NO: 5 - protein, 23 aa, Artificial Sequence
  Synthetic peptide
EQQLKYFLERFNEQLEDLFYQSS

SEQ ID NO: 6 - protein, 23 aa, Artificial Sequence
  Synthetic peptide
  X (12)..(12): X is A, N, or L
  Z (17)..(17): Z is D or Q
EQQLKYFLERFXRQLEZLFYQSS

SEQ ID NO: 7 - protein, 23 aa, Artificial Sequence
  Synthetic peptide
  X (12)..(12): X is A, N, or L
  Z (17)..(17): Z is D or Q
EQQLKYFLERFXKQLEZLFYQSS